(19) United States
(12) Patent Application Publication
(10) Pub. No.: US 2012/0149975 A1
(43) Pub. Date: Jun. 14, 2012

(54) LAPAROSCOPIC ADJUSTABLE GASTRIC BAND

(76) Inventor: Philip Cohen, Sterling, VA (US)

(21) Appl. No.: 13/314,678

(22) Filed: Dec. 8, 2011

Related U.S. Application Data
(60) Provisional application No. 61/421,372, filed on Dec. 9, 2010.

(51) Int. Cl. A61F 2/00

(52) U.S. Cl. 600/37

(57) ABSTRACT

An adjustable gastric band (AGB) positionable around a human stomach to limit the flow of food therethrough is provided with an improved structure for receiving and retaining suture from the stomach to the gastric restrictive device and thereby enabling a safer gastro-gastric plication while simultaneously preventing movement of the gastric fundus relative to the gastric restrictive device while additionally reducing the risk of tissue in-growth around and through the fenestrations, apertures or interstices of prior adjustable gastric band (AGB) structures for receiving and retaining sutures, which will render this embodiment safer in regard to the need for any potential revision procedures which may require relocating, removing or replacing the adjustable gastric bands in the future as well as enabling a safer gastro-gastricbuttress plication which will reduce the incidence of both “slippage” and “erosion.”

### CROSS REFERENCE TO RELATED APPLICATION

This application claims the benefit of U.S. Provisional Patent Application Ser. No. 61/421,372, entitled “LAPAROSCOPIC ADJUSTABLE GASTRIC BAND”, filed Dec. 9, 2010.

### BACKGROUND OF THE INVENTION

1. Field of the Invention

The present invention relates to adjustable gastric banding devices used in the treatment of obesity. When in use, adjustable gastric banding devices encircle the upper portion of a human stomach to form a stoma opening of reduced diameter so as to restrict food intake. More particularly, the present invention relates to improvements in adjustable gastric banding devices by reducing the incidence of slippage of the adjustable gastric band from its desired position as well as erosion of the adjustable gastric band into the lumen of the stomach. The present invention additionally provides a safer environment in the event of any need to relocate, remove or replace the adjustable gastric band in the future.

2. Description of the Related Art

Adjustable gastric banding devices are commonly placed inside the human abdomen laparoscopically, and secured in place around the upper portion of the stomach. They are usually secured in position with nonabsorbable, permanent sutures. In accordance with one currently employed methodology, the sutures are gastro-gastric sutures (or plication), which create anterolateral and posterolateral support for securing the adjustable gastric band (within the plication) with the patient's own stomach tissue in a manner frequently described by professionals skilled in the art as “belt loops”. In accordance with an alternate methodology, the sutures are applied in a manner providing for suturing the adjustable gastric band directly to the seromuscular layer of the stomach.

The “belt loops” are created by placing one bite of the suture material through the seromuscular layer of the gastric fundus inferior to the adjustable gastric band and a subsequent bite through the seromuscular layer of the newly created gastric pseudopouch superior to the properly placed adjustable gastric band. These two bites are then securely tied using one of several known methods including, but not limited to, intracorporeal laparoscopic knot tying, U-Clip™, SMART STITCH™ and/or ENDOSTITCH™. This “belt loop” construction incorporates the anterolateral and posterolateral portions of the implanted adjustable gastric band within the lumen created by the resulting tissue plication.

Although adjustable gastric bands have been very successful worldwide, there have been occasions when problems have occurred. For example, patients have encountered slippage or prolapse of the stomach up through the adjustable gastric band when previously located in its desired position. Patients have also been subject to erosion or migration of the adjustable gastric band through the full thickness of the underlying or adjacent gastric tissue. Both of these are undesirable and in some cases cause extreme medical complication to the patient who has had an adjustable gastric band implanted.

There are also inherent undesirabilities in the “belt loop” construct. Firstly, it is suspected that incorporating the adjustable gastric band within the lumen of the plication too snugly increases the incidence of erosion or migration of the adjustable gastric band into the lumen of the stomach. This may result from too tight initial placement of the gastro-gastric suture plication or as a result of tissue swelling resulting from patient regurgitation and/or vomiting subsequent to placement of the adjustable gastric band. Erosion or migration may also result from over-inflation of the adjustable gastric band balloon. Erosion or migration may also result from any combination of the factors described above.

Secondly, creating a “belt loop” construct which is too loose is thought to increase the incidence of slippage or prolapse of gastric fundus up through the properly positioned adjustable gastric band. Slippage or prolapse of the gastric fundus upwards through the properly positioned adjustable gastric band creates an unacceptably large pseudopouch which may accommodate a larger volume of food to be consumed before satiety is achieved, thereby interfering with the intended effective treatment of a patient's obesity. In extreme circumstances slippage or prolapse also creates a site for obstruction of the transit of food from the upper pseudopouch to the larger gastric lower compartment (body) which could result in a surgical emergency.

The suspected mechanism of these occurrences is the difficulty of creating a plication which is both roomy enough to accommodate future tissue swelling and/or balloon inflation, yet tight enough to prevent the gastric fundus from migrating up through a properly positioned adjustable gastric band as it has a tendency to do as a result of vomiting and/or overeating. A solution to these problems is effectively securing the adjustable gastric band in its proper position and, at the same time, eliminating the need to incorporate the adjustable gastric band within the tissue plication and thereby eliminating the nidus for possible erosion.

Prior attempts at achieving this goal involve the use of tabs, securing member(s), or holding mean(s) which extend from the backbone or belt of the adjustable gastric band and provide a mechanism for safely and securely receiving suture material at a distance away from the fragile balloon portion of the adjustable gastric band. All known prior devices have, so far, either intentionally or unintentionally, provided components which allow for some degree of tissue in-growth over time.

Those skilled in the art of caring for patients with adjustable gastric bands know that there are occasions when adjustable gastric bands need to be relocated, removed and/or replaced. In any of these revision scenarios, it is highly desirable to have the fewest possible attachments of human tissue to the adjustable gastric band.

The present invention overcomes the deficiencies found in currently available adjustable gastric bands by providing an adjustable gastric band with the fewest, most streamlined, yet secure sites for suture attachment between the gastric fundus and the adjustable gastric band. This results in a higher degree of patient safety by limiting the amount and tenacity of scar tissue able to incorporate itself within the superficial interstices of the adjustable gastric band.

### SUMMARY OF THE INVENTION

It is, therefore, an object of the present invention to provide a gastric band including a balloon and a belt positioned about an outer circumference of the balloon. The belt includes a first end and a second end. The respective first end and second end of the belt are provided with mating latching members allowing for selective and secure placement about a stomach. The belt also includes an inferior side edge from which a suture receiving strip extends, the suture receiving strip including a plurality of dimples.

It is also an object of the present invention to provide a gastric band wherein the suture receiving strip extends along the inferior side edge of the belt along only anterolateral, lateral and posterolateral portions of the inferior side edge of the belt.

It is another object of the present invention to provide a gastric band wherein the suture receiving strip is a smooth strip of silicone.

It is a further object of the present invention to provide a gastric band wherein the suture receiving strip includes 3-8 dimples located along a horizontal plane, located midway between superior and inferior borders of the suture receiving strip.

It is also an object of the present invention to provide a method including securing the gastric band to a position on the stomach with the suture receiving strip extending along a wall of the stomach, forming a plication by drawing tissue toward the suture receiving strip, and securing the plication.

It is another object of the present invention to provide a method wherein the step of securing includes positioning the suture receiving strip along a lateral wall of the stomach.

It is a further object of the present invention to provide a method wherein the suture receiving strip is positioned between an anterior wall and a posterior wall of the stomach.

It is also an object of the present invention to provide a method wherein the step of forming the plication includes drawing redundant fundal tissue upward and toward the suture receiving strip.

It is a further object of the present invention to provide a method wherein the step of forming the plication includes gathering and securing anterolateral, lateral and posterolateral gastric fundus.

It is also an object of the present invention to provide a method wherein the step of securing includes applying sutures through both the tissue and the suture receiving strip.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a side frontal plan view of the present adjustable gastric band with the adjustable gastric band latched in a circular configuration.

FIG. 2 is a frontal plan flattened view with the adjustable gastric band unlatched and laid in an extended configuration.

FIG. 3 is a rear plan view of the adjustable gastric band as it would appear when viewed posteriorly on the stomach.

FIG. 4 is a plan left side view of the adjustable gastric band as it would appear when viewed laterally on the stomach.

FIGS. 5, 6 and 7 are frontal views of the stomach along the coronal or fontal plane disclosing steps for implementing the formation of the buttressplication along the anterolateral, lateral and posterolateral portion of the stomach through the utilization of the present adjustable gastric band.

FIGS. 8 and 9 are respectively an anterior and lateral view(s) of the stomach respectively along the coronal (or frontal) plane and the sagittal plane with the present adjustable gastric band secured to the gastro-gastric buttress plication thereto.

### DESCRIPTION OF THE PREFERRED EMBODIMENT

The detailed embodiment of the present invention is disclosed herein. It should be understood, however, that the disclosed embodiment is merely exemplary of the invention, which may be embodied in various forms. Therefore, the details disclosed herein are not to be interpreted as limiting, but merely as a basis for teaching one skilled in the art how to make and/or use the invention.

Referring to the various figures, and in accordance with a preferred embodiment of the present invention, the present adjustable gastric band 10 creates an improved and safer environment for securing the adjustable gastric band 10 in its desired position relative to the stomach 100. This is achieved by providing the adjustable gastric band 10 with a suture receiving strip 12 along the inferior or bottom side edge 24 thereof. Specifically, the suture receiving strip 12 extends along a portion of the length of the adjustable gastric band 10 such that the suture receiving strip 12 is positioned along the anterolateral, lateral and posterolateral portion 102, 104, 106 of the stomach 100 when properly positioned on the stomach 100. The suture receiving strip 12 is provided with a plurality of dimples 16 used in guiding sutures therethrough for securing the adjustable gastric band 10 in position. These dimples 16 are of minimal size, just large enough to accommodate a suture needle of optimal size, approximately 1 mm or less in diameter, yet not too large which may allow for unnecessary tissue ingrowth. It should be appreciated the use of the term “dimple” is meant to refer to a female recess formed in the surface of the suture receiving strip 12. The recess functions as a guide for a suture needle and is therefore formed with a conical, concave shape guiding the needle to the thinnest portion of the suture receiving strip 12 where it may be pushed therethrough.

This present adjustable gastric band 10 eliminates the need for the undesirable anterolateral, lateral and posterolateral gastro-gastric plication (“belt loop” construct) of the anterior, lateral and posterior gastric fundus to the newly created gastric pseudopouch.

By eliminating the need for the gastro-gastric plication of the gastric fundus (“belt loop” construct), the present adjustable gastric band 10 eliminates the need for the anterolateral, lateral and posterolateral “tunnel” created by folding the gastric fundus over an adjustable gastric band. As discussed above in the Background of the Invention, this “tunnel” has been implicated in the occurrence of late erosion of the adjustable gastric band into the lumen of the stomach. Eliminating the need for this problematic gastro-gastric tunnel provides for an inherent increase in safety.

The present adjustable gastric band 10 also includes a safer environment than prior adjustable gastric bands which provide fenestrated appendices for direct attachment of suture material from the seromuscular layer of the stomach, directly to the fenestrated appendices of the adjustable gastric band. The present adjustable gastric band 10 thereby minimizes tissue in-growth common in currently existing adjustable gastric bands employing tabs, securing member(s) or holding element(s) as discussed above. By minimizing tissue in-growth, the present adjustable gastric band 10 provides the additional ability to secure the adjustable gastric band in its intended location with minimal difficulty in relocating, removing or replacing the adjustable gastric band in the future due to tissue in-growth should it become necessary.

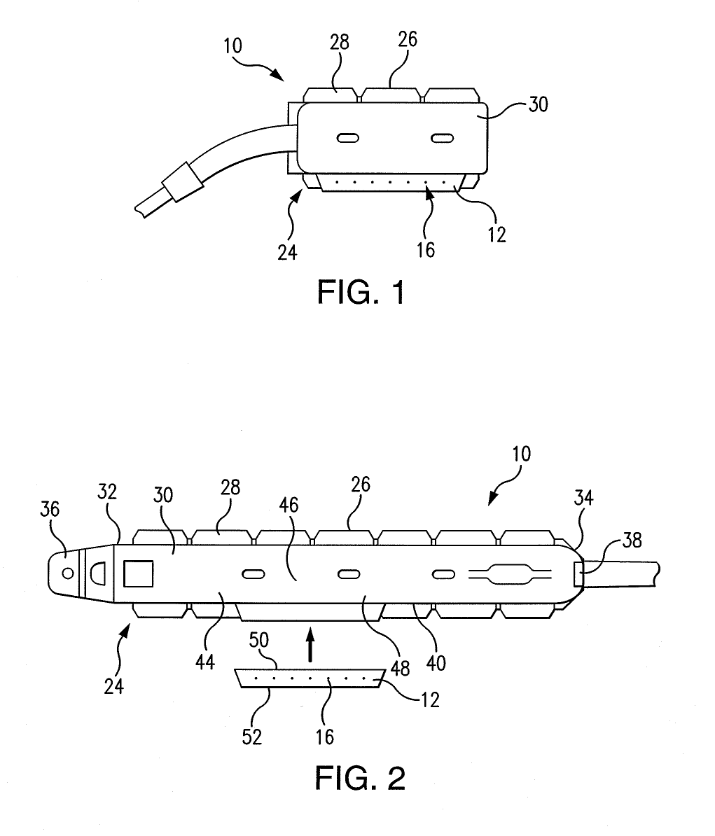

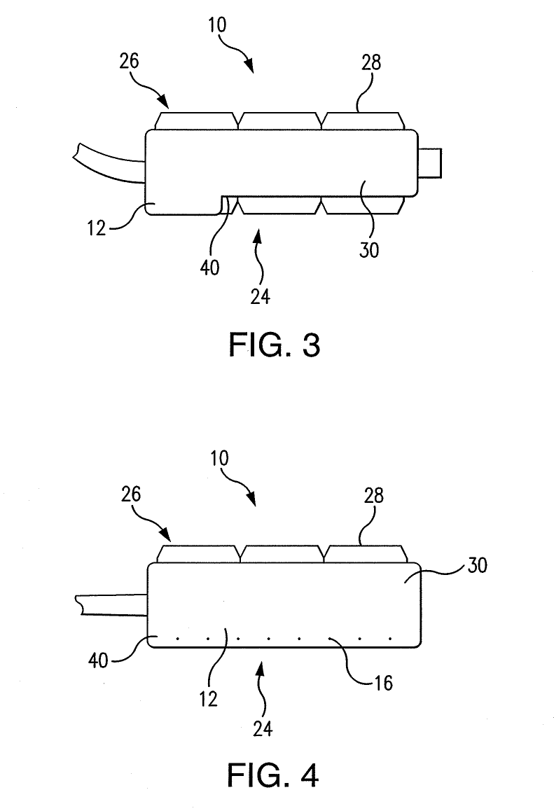

Referring now to FIGS. 1 to 4, the present adjustable gastric band 10 has an inferior (or bottom) side edge 24 and a superior (or top) side edge 26. The adjustable gastric band 10 is further provided with a central balloon 28 shaped and dimensioned for expansion and contraction in a manner well known to those skilled in the art. As such, the balloon 28 in accordance with the present invention may take a wide variety of known and future constructions without departing from the spirit of the present invention.

Positioned about the outer circumference of the balloon 28 is a supporting belt 30. The belt 30 includes a first end 32 and second end 34. The respective first and second ends 32, 34 of the belt 30 are provided with mating latching members 36, 38 allowing for selective and secure placement about the stomach in a manner well known to those skilled in the art. It is contemplated the belt 30, in accordance with the present invention, may take a wide variety of known constructions without departing from the spirit of the present invention.

Extending from the inferior side edge 24 of the gastric band 10, and in particular, from the inferior side edge 40 of the belt 30, is a suture receiving strip 12 having a plurality of dimples 16 formed therein. More specifically, the suture receiving strip 12 extends along the inferior side edge 24 along only the anterolateral, lateral and posterolateral portions 44, 46, 48 of the inferior side edge 40 of the belt 30 (and, ultimately, the gastric band 10). As those skilled in the art will certainly appreciate, the anterolateral portion 44, lateral portion 46 and posterolateral portion 48 refer to the positioning of the adjustable gastric band 10 when it is properly received about the stomach. Referring to FIG. 2, the lateral portion 46 of the belt 30 would generally correspond to the central portion of the belt 30 when viewed with the adjustable gastric band 10 laid out as shown in FIG. 2, while the anterolateral and posterolateral portions 44, 48 are those sections to the adjacent sides of the central portion. The provision of a suture receiving strip 12 with a plurality of dimples 16 formed therein provides limited opportunities for tissue in-growth while still providing effective mechanisms for securing the adjustable gastric band 10 in its preferred position on the stomach.

The ability to secure the adjustable gastric band 10 about the stomach is facilitated by the provision of the suture receiving strip 12 as discussed herein. The suture receiving strip 12 is made of the same or similar material as used in the manufacture of the current, conventional adjustable gastric band nonextensible silicone belt 30 or backbone. It will be appreciated the material will ultimately be determined based upon a balance between durability (ability to withstand the forces generated by violent vomiting) and flexibility (ability to stretch and return to original shape as a result of violent vomiting). The suture receiving strip 12 is of a dimension and flexibility which allows ease of introduction into the abdomen through a conventional 15 mm trocar or cannula. In addition, the suture receiving strip 12 is of the lowest profile in the dimension of the adjustable gastric band 10 which, when initially placed at the time of surgery, may need to pass through the anatomic tunnel posterior to the stomach and inferior to the crura of the diaphragm. In accordance with a preferred embodiment, the suture receiving strip 12 is a small, smooth strip of silicone (or similar material), located anterolaterally, laterally and posterolaterally when the adjustable gastric band 10 is situated in its usual and preferred position about the stomach.

As discussed above, the suture receiving strip 12 is formed with a plurality of dimples 16. That is, the strip 12 is dimpled but not penetrated until a suture has been passed therethrough. The dimples 16 assist the surgeon in placing the suture needle by functioning as a targeting mark for optimal safe placement of suture. The suture receiving strip 12 preferably has 3-8 dimples located along a horizontal plane, located midway between the superior and inferior borders 50, 52 of the suture receiving strip 12. By providing dimples 16 for guiding the passage of sutures and eliminating fenestrations, apertures, interstices or other perforate material in the adjustable gastric band 10, in particular, the suture receiving strip 12, the present invention avoids the undesirable effect of subsequent tissue in-growth after placement of the adjustable gastric band 10. The presence of tissue in-growth would make subsequent revision procedures more difficult and potentially more dangerous for the patient. As discussed above, the material of the suture receiving strip 12 is resilient; allowing it to close around sutures passed therethrough. The closure of the suture receiving strip 12 material about holes formed by the passage of a suture needle therethrough minimizes and may substantially eliminate the possibility for tissue in-growth.

The suture receiving strip 12 is positioned along the lower section or border of a specific segment of the band 10, namely from the anterolateral, lateral and posterolateral portions 44 46, 48 of the inferior side edge 40 of the belt 30 which eliminates the need for an unnecessarily larger tunnel, posterior to the stomach (as referred to in U.S. Pat. No. 5,910,149).

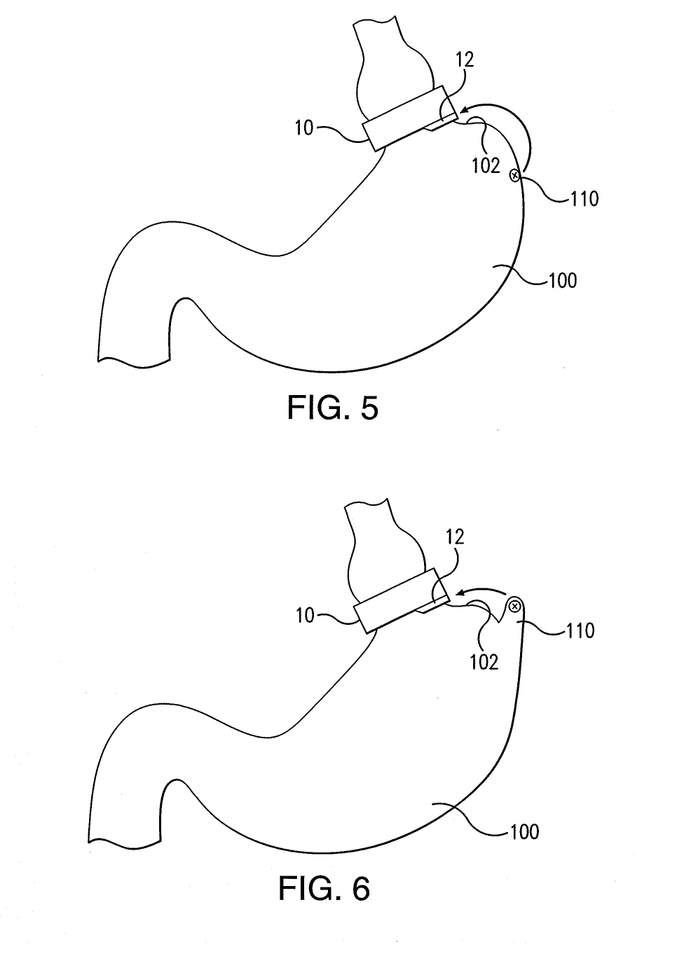

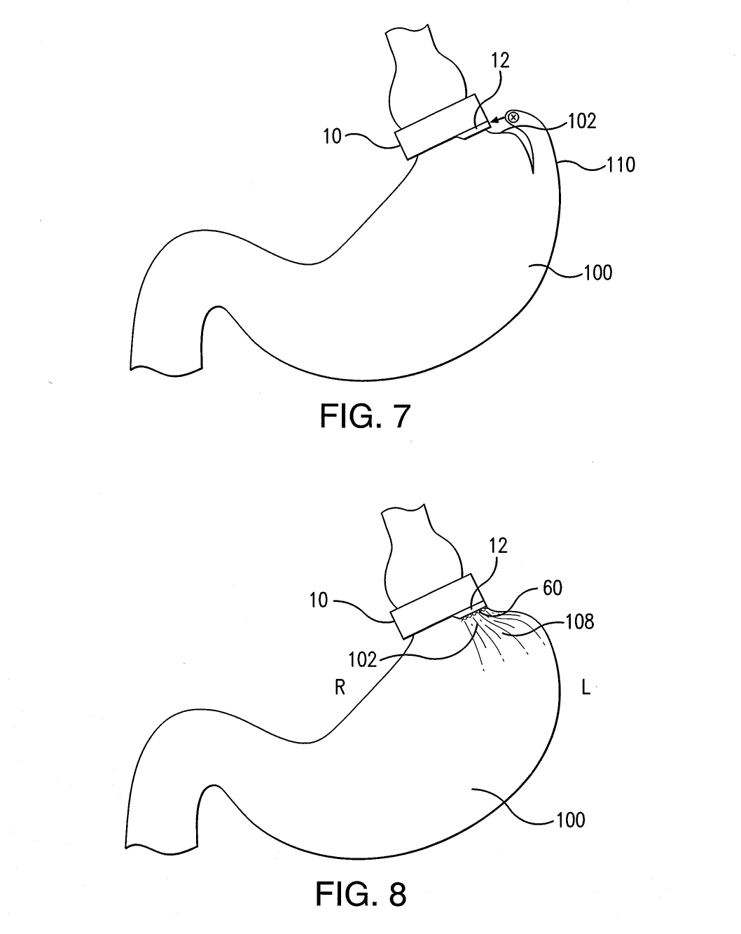

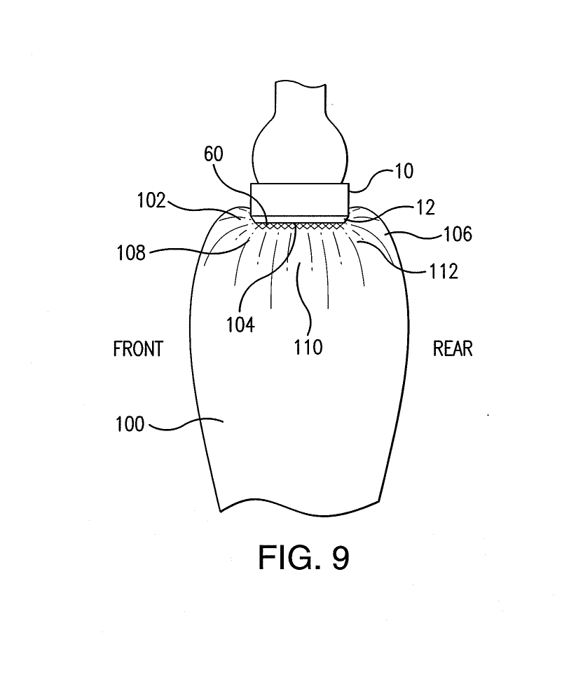

Use of the present gastric band 10 allows for the possibility of employing anti-slip stitches (gathering the anterior, lateral and posterior gastric fundus inferior to the adjustable gastric band 10). In accordance with the present invention, this procedural step is improved by the positioning of the suture receiving strip 12 along the inferior side edge 24 of the adjustable gastric band 10 and only extending the suture receiving strip 12 along the anterolateral, lateral and posterolateral portions 44, 46, 48 on the belt 30 of the band 10. By locating the placement of sutures used in gathering the anterior, lateral and posterior gastric fundus 108, 110, 112 and to a location just inferior to the properly placed adjustable gastric band 10, and only from the anterolateral, lateral and posterolateral portions 44,46, 48 of the band 10, the present adjustable gastric band 10 creates a more desirable buttress of gastric tissue inferior to the stoma (see FIGS. 5-9) of the adjustable gastric band 10. This results in a decrease in the ability of the gastric fundus to prolapse up through the properly positioned adjustable gastric band 10.

The present adjustable gastric band 10, with its more desirable buttress of gastric tissue inferior to the stoma, eliminates the problematic gastro-gastric tunnel which incorporates the adjustable gastric band within it. The present adjustable gastric band 10 is securely positioned employing a buttress or similar gathering (or imbricating) of stomach fundus 108, 110, 112, inferior to the properly located gastric band 10 as shown in FIGS. 5-9. This construct retains the important ability of securing the adjustable gastric band 10 in position and preventing the fundus from prolapsing up through the adjustable gastric band 10.

In accordance with a preferred methodology for utilization of the present adjustable gastric band 10, a procedure for the formation of aplication of the fundus 108, 110, 112 at a location inferior to the properly positioned adjustable gastric band 10 and along the anterolateral, lateral and posterolateral portions 44, 46, 48 of the properly positioned adjustable gastric band 10 is disclosed with reference to FIGS. 5 to 9. In particular, and after the present adjustable gastric band 10 is secured to its optimal position on the upper portion of the stomach 100 with the suture receiving strip 12 extending along the lateral wall of the stomach 100 between the anterior wall and the posterior walls of the stomach as shown with reference to FIGS. 5-9, a plication is formed by drawing redundant fundal tissue 108, 110, 112 (2-5 cm inferior to the adjustable gastric band 10) upward and toward the suture receiving strip 12 of the present adjustable gastric band 10. More particularly, anterolateral, lateral and posterolateral gastric fundus 108, 110, 112 are gathered and secured to the suture receiving strip 12.

As shown with reference to FIGS. 5-9, once the tissue of the anterolateral, lateral and posterolateral gastric fundus is pulled upwardly toward the suturing strip, a plication is formed inferior to the adjustable gastric band 10. Thereafter, the plication is securely held in position through the application of sutures 60 through both the tissue and the suture receiving strip 12.

Additionally, the adjustable gastric band 10, having an inferior suture receiving strip 12 attached thereto, can be utilized during a “greater curvature plication”, pioneered as described by Dr. Ramos [OBES SURG (2010) 20:913-918], of or as a “gastric sleeve plication,” as described by Schauer [Laparoscopic Gastric Plication for the Treatment of Severe Obesity. Brethauer, Harris, Kroh, Schauer. Surgery for Obesity and Related Diseases—11 Oct. 2010 (10.1016/j.soard.2010.09.023)]. The gastric band 10 can be placed around the imbricated tissue and the tissue can then be sewn to the suture receiving strip 12 located on the gastric band 10.

While the preferred embodiments have been shown and described, it will be understood that there is no intent to limit the invention by such disclosure, but rather, is intended to cover all modifications and alternate constructions falling within the spirit and scope of the invention.

1. A gastric band, comprising:
a balloon and a belt positioned about an outer circumference of the balloon; the belt includes a first end and a second end, the respective first end and second end of the belt are provided with mating latching members allowing for selective and secure placement about a stomach;
the belt also includes an inferior side edge from which a suture receiving strip extends, the suture receiving strip including a plurality of dimples.

2. The gastric band according to claim 1, wherein the suture receiving strip extends along the inferior side edge of the belt along only anterolateral, lateral and posterolateral portions of the inferior side edge of the belt.

3. The gastric band according to claim 1, wherein the suture receiving strip is a smooth strip of silicone.

4. The gastric band according to claim 1, wherein the suture receiving strip includes 3-8 dimples located along a horizontal plane, located midway between superior and inferior borders of the suture receiving strip.

5. A method for applying a gastric band to a stomach, the gastric band including a balloon and a belt positioned about an outer circumference of the balloon, the belt includes a first end and a second end, the respective first end and second end of the belt are provided with mating latching members allowing for selective and secure placement about the stomach, the belt also includes an inferior side edge from which a suture receiving strip extends, the suture receiving strip including a plurality of dimples, the method comprising:
securing gastric band to a position on the stomach with the suture receiving strip extending along a wall of the stomach;
forming a plication by drawing tissue toward the suture receiving strip; and
securing the plication.

6. The method according claim 5, wherein the step of securing includes positioning the suture receiving strip along a lateral wall of the stomach.

7. The method according claim 6, wherein the suture receiving strip is positioned between an anterior wall and a posterior walls of the stomach.

8. The method according claim 5, wherein the step of forming the plication includes drawing redundant fundal tissue upward and toward the suture receiving strip.

9. The method according claim 8, wherein the step of forming the plication includes gathering and securing anterolateral, lateral and posterolateral gastric fundus.

10. The method according claim 5, wherein the step of securing includes applying sutures through both the tissue and the suture receiving strip.
